(19) United States
(12) Patent Application Publication
(10) Pub. No.: US 2013/0261084 A1
(43) Pub. Date: Oct. 3, 2013

(54) METHOD OF IMPROVING RESPONSE TO CHEMOTHERAPY BY ADMINISTERING A PHOSPHOLIPID TOGETHER WITH CHEMOTHERAPEUTIC AGENTS, AND FORMULATIONS THEREFOR

(71) Applicant: Robert L. Saxe, New York, NY (US)

(72) Inventor: Robert L. Saxe, New York, NY (US)

(21) Appl. No.: 13/432,426

(22) Filed: Mar. 28, 2012

(51) Int. Cl. A61K 31/685; A61P 35/00

(52) U.S. Cl. 514/78

(57) ABSTRACT

Methods and formulations for improving efficacy of chemotherapeutic treatment. The formulation includes at least one chemotherapeutic agent and at least one phospholipid or a material comprising the same. The phospholipid facilitates entry of the at least one chemotherapeutic agent into cells of an afflicted individual. A sufficient dosage of a blood-thinning agent is optionally included to mitigate risk of developing blood clots attributable to administration of the chemotherapeutic agent(s). The chemotherapeutic agent(s) and the phospholipid(s), or the material containing the phospholipid(s), are administered in any order but in sufficient time proximity to one another such that the effect of the phospholipid(s) of facilitating entry of the chemotherapeutic agent(s) into the cells at least partially overlaps with the therapeutic effect of the chemotherapeutic agent(s).

### TECHNICAL FIELD

The present invention relates to methods and formulations for improving the efficacy of chemotherapeutic therapy used in treating diseases, e.g., cancers such as multiple myeloma, and/or other conditions in a human being or animal, involving administration of at least one phospholipid or a material comprising at least one said phospholipid and, optionally, a blood thinner such as aspirin, in combination with one or more chemotherapeutic agents to those afflicted with the disease/condition.

### BACKGROUND OF THE INVENTION

Chemotherapy is defined as the treatment of a disease or condition, commonly but not necessarily a cancer, with an antineoplastic drug or a combination of such drugs in a standardized treatment regimen. Various types or classes of chemotherapeutic agents, in a variety of combinations and in various strengths are used in a variety of regimens to aid patients suffering from diseases such as cancers, e.g., multiple myeloma, and numerous other conditions as well. The degree to which chemotherapy can benefit a patient depends on the patient, the patient's medical condition, the disease or condition afflicting the patient and other factors. Of course, a highly desirable outcome of treatment with chemotherapy would be for the patient to achieve complete remission from the disease. However, even in the absence of complete remission, any significant improvement in the patient's medical condition, as evidenced for example, by the patient's blood test results and physical well being, would be welcome and useful.

The inventor of the methods and formulations described and claimed herein has been afflicted with multiple myeloma, and has obtained complete and very rapid remission therefrom, whereupon he has concluded that the methods and formulations described herein were helpful, if not significantly responsible in achieving such rapid remission.

In cases where chemotherapy is intended to destroy abnormal cells, its effectiveness can be less than desired if (a) certain of the abnormal cells are located in places in an individual's body where the chemotherapeutic agent(s) cannot or can only with difficulty, reach them or (b) the chemotherapeutic agent(s) cannot overcome or has/have difficulty overcoming the defenses of the abnormal cells, e.g., if the chemotherapy cannot easily penetrate the membrane of the cell wall.

An amphiphile is a term describing a chemical compound possessing both hydrophilic (water-loving, polar) and lipophilic (fat-loving) properties. Such a compound is called amphiphilic or amphipathic. Common amphiphilic compounds include, but are not limited to, phospholipids, which are a class of amphiphilic molecules. It is, furthermore, well known that phospholipids are important components of biological membranes. The amphiphilic nature of these molecules defines the manner in which they form such membranes. They arrange themselves into bilayers, by positioning their polar groups towards the surrounding aqueous medium, and their lipophilic chains towards the inside of the bilayer, defining a non-polar region between two polar ones.

The two major sub-classes of phospholipids are phosphatides and sphingosines. Phosphatides are a type of phospholipid made up mainly of glycerol, fatty acids and phosphate. They are the type of lipid that makes up the bulk of the phospholipids found in cell membranes.

A common source of phospholipids is lecithin. It has been reported that lecithin is found in all living cells as a major component comprising the phospholipid portion of the cell membrane that regulate the nutrients entering and exiting the cell. (C. Lawhon, “Lecithin Supplements Effectiveness in Weight Loss” www.vanderbiltedu/ans/psychology/health_psychology/LECITHIN_SUPPLEMENT) Lecithin comprises any group of yellow-brownish fatty substances occurring in animal and plant tissues, and in egg yolk, composed of phosphoric acid, choline, fatty acids, glycerol, glycolipids, triglycerides and phospholipids such as phosphatidyl choline, phosphatidylethanolamine and phosphatidylinositol. As used herein the term lecithin has the meaning set forth above, together with all minor useful variations thereof. (see, e.g., wikipedia.org/wiki/Lecithin) Containers of lecithin granules can be purchased as a dietary supplement in many health food stores. So far as the inventor's knowledge of this field is concerned, there has heretofore not been any report that lecithin or any of its components is useful in enhancing the effectiveness of chemotherapy.

### SUMMARY OF THE INVENTION

In one embodiment the invention is directed to a method for improving efficacy of a chemotherapeutic treatment of a disease or condition. The method comprises administering a therapeutic amount of at least one chemotherapeutic agent to an individual afflicted with a disease or condition that is treatable by the at least one chemotherapeutic agent; and also administering to the individual at least one phospholipid or a material containing at least one phospholipid, wherein the at least one phospholipid is administered in an amount sufficient to facilitate entry of the at least one therapeutic agent into cells of the individual afflicted by the disease or condition. The above-described steps may be performed in any order but in sufficient time proximity to one another such that the effect of the at least one phospholipid in facilitating entry of the at least one chemotherapeutic agent into cells of the afflicted individual at least partially overlaps with the therapeutic effect of the at least one chemotherapeutic agent.

In another embodiment the invention also is directed to a method for improving efficacy of a chemotherapeutic agent in treating a disease or condition wherein the method comprises administering a therapeutic amount of at least one chemotherapeutic agent to an individual afflicted with a disease or condition that is treatable by the at least one chemotherapeutic agent; administering to the individual at least one phospholipid or a material containing at least one said phospholipid, wherein the at least one phospholipid is administered in an amount sufficient to facilitate entry of the at least one therapeutic agent into cells of the individual negatively affected by the disease or condition; and additionally administering a sufficient dosage of a blood-thinning agent to the individual to mitigate risk of said individual developing blood clots caused due to administration of the at least one chemotherapeutic agent. As in the embodiment described in the paragraph above, the administration steps may be performed in any order but in sufficient time proximity to one another such that the effect of the at least one phospholipid in facilitating entry of the at least one chemotherapeutic agent into cells of the afflicted individual at least partially overlaps with the therapeutic effect of the at least one chemotherapeutic agent.

In a further embodiment the invention is directed to a formulation that improves efficacy of chemotherapeutic treatment of an individual afflicted with a condition or disease treatable with one or more chemotherapeutic agents. The formulation comprises at least one chemotherapeutic agent capable of treating a disease or condition with which the individual is afflicted and at least one phospholipid or a material containing at least one phospholipid, wherein the at least one phospholipid acts to facilitate entry of the at least one chemotherapeutic agent into cells of the afflicted individual. The at least one chemotherapeutic agent and the at least one phospholipid or the material containing the at least one phospholipid may be administered in any order but in sufficient time proximity to one another that the effect of the at least one phospholipid of facilitating entry of the at least one chemotherapeutic agent into cells of the afflicted individual at least partially overlaps with the therapeutic effect of the at least one chemotherapeutic agent.

In another embodiment the invention also is directed to another formulation that improves efficacy of chemotherapeutic treatment of an individual afflicted with a condition or disease treatable with one or more chemotherapeutic agents. The formulation comprises at least one chemotherapeutic agent capable of treating a disease or condition with which the individual is afflicted; at least one phospholipid or a material containing at least one phospholipid, wherein the at least one phospholipid acts to facilitate entry of the at least one chemotherapeutic agent into cells of the afflicted individual; and a sufficient dosage of a blood-thinning agent to mitigate risk to the individual of developing blood clots attributable to administration of the at least one chemotherapeutic agent. The at least one chemotherapeutic agent and the at least one phospholipid or the material containing the at least one phospholipid may be administered in any order but in sufficient time proximity to one another that the effect of the at least one phospholipid of facilitating entry of the at least one chemotherapeutic agent into cells of the afflicted individual at least partially overlaps with the therapeutic effect of the at least one chemotherapeutic agent.

The administration of the chemotherapeutic agents and the one or more phospholipids may be performed in any order but such administration must take place in sufficient time proximity such that the effect of the phospholipid in facilitating entry of the chemotherapeutic agent(s) into the cell and the therapeutic effect of the chemotherapeutic agent(s) on the abnormal cells at least partially overlap.

Accordingly, although the inventor does not wish to be bound by theory, it is believed that a phospholipid or a material such as lecithin comprising a phospholipid is capable, i.e., by virtue of the presence of the phospholipid, of enhancing the effectiveness of chemotherapy in two ways. First, by acting as an excellent dispersing agent and emulsifier it is believed to make a patient's blood more uniform, i.e., less lumpy, and to thereby enable the blood to reach some abnormal cells that might otherwise be difficult to reach. And secondly, the phospholipid may offer chemotherapeutic agents easier access to enter abnormal cells and thus give such agents an opportunity to destroy the abnormal cells from within. Lecithin, because of its widespread presence in the human body as a cell membrane component may be especially useful in this regard; however, any phospholipid operating as described above, i.e., as a dispersing agent and an emulsifier, and which facilitates entry of chemotherapeutic agents into the subject's cells, is deemed to fall within the scope of the present invention.

Optionally, to enhance the flow of blood by lowering its viscosity, a blood-thinning agent such as, but not limited to, aspirin or pycnogenol may also be administered as part of the presently described method/composition.

### DETAILED DESCRIPTION

As indicated above the discoveries described herein are directed to methods and formulations for improving efficacy of a chemotherapeutic treatment of a disease or condition in an individual afflicted with such disease or condition. The method comprises, in one embodiment, administering to the individual a therapeutic amount of at least one chemotherapeutic agent as well as at least one phospholipid or a material containing the at least one phospholipid wherein the at least one phospholipid is administered in an amount sufficient to facilitate entry of the at least one chemotherapeutic agent into cells of the individual afflicted with the disease or condition. Optionally, in a further embodiment, the method further comprises administering a sufficient amount of a blood-thinning agent to the individual to mitigate risk of the individual's developing blood clots attributable to administration of the at least one chemotherapeutic agent. The administration of the chemotherapeutic agent and the phospholipid or material containing at least one phospholipid may occur in any order, but in sufficient time proximity that the effect of the at least one phospholipid in facilitating entry of the at least one chemotherapeutic agent into cells of the afflicted individual at least partially overlaps with the therapeutic effect of the at least one chemotherapeutic agent.

In one embodiment of the above-described method, the material containing the at least one phospholipid is lecithin. In another embodiment the disease or condition with which the individual is afflicted may be a cancer treatable by the at least one chemotherapeutic agent. In a further embodiment the cancer is a hematological malignancy. In a still further embodiment the hematological malignancy is multiple myeloma. In embodiments where the blood-thinning agent is administered, useful agents may include, but are not limited to, aspirin and pycnogenol. In another embodiment the phospholipid or the material containing the at least one phospholipid is formulated into a dosage form including, but not limited to, tablets, capsules, vegetable capsules, an injectable, a trans-dermal patch and liquid solutions formulated for drinking or for infusion.

A further embodiment constitutes a formulation that acts to improve efficacy of chemotherapeutic treatment of an individual afflicted with a condition or a disease that is treatable with one or more chemotherapeutic agents. The formulation comprises at least one chemotherapeutic agent capable of treating a disease or condition with the individual is afflicted and at least one phospholipid or a material comprising at least one said phospholipid, wherein the at least one phospholipid acts to facilitate entry of the at least one chemotherapeutic agent into cells of the afflicted individual. Optionally, in a further embodiment, the formulation further comprises a sufficient amount of a blood-thinning agent to the individual to mitigate risk of developing blood clots attributable to administration of the at least one chemotherapeutic agent. The chemotherapeutic agent and the phospholipid or material containing at least one phospholipid may be administered in any order, but in sufficient time proximity that the effect of the at least one phospholipid in facilitating entry of the at least one chemotherapeutic agent into cells of the afflicted individual at least partially overlaps with the therapeutic effect of the at least one chemotherapeutic agent.

In one embodiment of the above-described formulation the material containing the at least one phospholipid is lecithin. In another embodiment the disease or condition with which the individual is afflicted may be a cancer treatable by the at least one chemotherapeutic agent. In a further embodiment the cancer is a hematological malignancy. In a still further embodiment the hematological malignancy is multiple myeloma. In embodiments where the blood-thinning agent is administered, useful agents may include, but are not limited to, aspirin and pycnogenol. In another embodiment the phospholipid or the material containing the at least one phospholipid is formulated into a dosage form including, but not limited to, tablets, capsules, vegetable capsules, an injectable, a trans-dermal patch and liquid solutions formulated for drinking or for infusion. Furthermore, the formulation according to the invention may be administered, i.e., in the appropriate dosage form, orally, intravenously, intramuscularly, subcutaneously, via implant, transmucosally, transdermally, rectally, nasally, by depot injection or by inhalation and pulmonary absorption. The formulation may be administered once as a time-release formulation, a plurality of times, or over one or more extended periods. Dosage levels of, e.g., the chemotherapeutic agents, are reported in the literature and are well-known among those having at least an ordinary level of skill in the relevant field. With respect to such dosages, the attending clinician should monitor individual patient response and adjust the dosage accordingly.

As used herein, a chemotherapeutic treatment of a disease or condition should be taken to refer to administering one or more compositions, drugs, formulations, etc., that are or may be used in a chemotherapy regimen to treat a disease or condition such as, but not limited to, a cancer. Such treatments typically, but not exclusively, utilize one or more chemotherapeutic (or chemotherapy) agents selected from among those listed below in Table 1. The list is reprinted from the Internet web site of Chemocare, found at Chemocare.com (www.chemocare.com/bio/). The list in Table 1 of chemotherapeutic agents is not meant to be exhaustive but rather it is provided to illustrate a number of such agents useful in the methods and formulations described and claimed herein. As used herein, “improving efficacy of a chemotherapeutic treatment of a disease or condition” should be taken to mean obtaining an increased or enhanced effect attributable to administration of one or more chemotherapeutic agents in comparison to the effect of such agent(s) that may otherwise be obtained in the absence of the phospholipid component in the methods and formulations described herein.

TABLE 1

1
13-cis-Retinoic Acid
2
2-CdA
2-Chlorodeoxyadenosine
5
5-Azacitidine
5-Fluorouracil
5-FU
6
6-Mercaptopurine
6-MP
6-TG
6-Thioguanine
A
Abraxane
Accutane ®
Actinomycin-D
Adriamycin ®
Adrucil ®
Afinitor ®
Agrylin ®
Ala-Cort ®
Aldesleukin
Alemtuzumab
ALIMTA
Alitretinoin
Alkaban-AQ ®
Alkeran ®
All-transretinoic Acid
Alpha Interferon
Altretamine
Amethopterin
Amifostine
Aminoglutethimide
Anagrelide
Anandron ®
Anastrozole
Arabinosylcytosine
Ara-C
Aranesp ®
Aredia ®
Arimidex ®
Aromasin ®
Arranon ®
Arsenic Trioxide
Arzerra ™
Asparaginase
ATRA
Avastin ®
Azacitidine
B
BCG
BCNU
Bendamustine
Bevacizumab
Bexarotene
BEXXAR ®
Bicalutamide
BiCNU
Blenoxane ®
Bleomycin
Bortezomib
Busulfan
Busulfex ®
C
C225
Calcium Leucovorin
Campath ®
Camptosar ®
Camptothecin-11
Capecitabine
Carac ™
Carboplatin
Carmustine
Carmustine Wafer
Casodex ®
CC-5013
CCI-779
CCNU
CDDP
CeeNU
Cerubidine ®
Cetuximab
Chlorambucil
Cisplatin
Citrovorum Factor
Cladribine
Cortisone
Cosmegen ®
CPT-11
Cyclophosphamide
Cytadren ®
Cytarabine
Cytarabine Liposomal
Cytosar-U ®
Cytoxan ®
D
Dacarbazine
Dacogen
Dactinomycin
Darbepoetin Alfa
Dasatinib
Daunomycin
Daunorubicin
Daunorubicin Hydrochloride
Daunorubicin Liposomal
DaunoXome ®
Decadron
Decitabine
Delta-Cortef ®
Deltasone ®
Denileukin Diftitox
DepoCyt ™
Dexamethasone
Dexamethasone Acetate
Dexamethasone Sodium Phosphate
Dexasone
Dexrazoxane
DHAD
DIC
Diodex
Docetaxel
Doxil ®
Doxorubicin
Doxorubicin Liposomal
Droxia ™
DTIC
DTIC-Dome ®
Duralone ®
E
Efudex ®
Eligard ™
Ellence ™
Eloxatin ™
Elspar ®
Emcyt ®
Epirubicin
Epoetin Alfa
Erbitux
Erlotinib
Erwinia L-asparaginase
Estramustine
Ethyol
Etopophos ®
Etoposide
Etoposide Phosphate
Eulexin ®
Everolimus
Evista ®
Exemestane
F
Fareston ®
Faslodex ®
Femara ®
Filgrastim
Floxuridine
Fludara ®
Fludarabine
Fluoroplex ®
Fluorouracil
Fluorouracil (cream)
Fluoxymesterone
Flutamide
Folinic Acid
FUDR ®
Fulvestrant
G
G-CSF
Gefitinib
Gemcitabine
Gemtuzumab ozogamicin
Gemzar
Gleevec ™
Gliadel ® Wafer
GM-CSF
Goserelin
Granulocyte - Colony Stimulating Factor
Granulocyte Macrophage Colony Stimulating Factor
H
Halotestin ®
Herceptin ®
Hexadrol
Hexalen ®
Hexamethylmelamine
HMM
Hycamtin ®
Hydrea ®
Hydrocort Acetate ®
Hydrocortisone
Hydrocortisone Sodium Phosphate
Hydrocortisone Sodium Succinate
Hydrocortone Phosphate
Hydroxyurea
I
Ibritumomab
Ibritumomab Tiuxetan
Idamycin ®
Idarubicin
Ifex ®
IFN-alpha
Ifosfamide
IL-11
IL-2
Imatinib mesylate
Imidazole Carboxamide
Interferon alfa
Interferon Alfa-2b (PEG Conjugate)
Interleukin-2
Interleukin-11
Intron A ® (interferon alfa-2b)
Iressa ®
Irinotecan
Isotretinoin
Ixabepilone
Ixempra ™
K
Kidrolase (t)
L
Lanacort ®
Lapatinib
L-asparaginase
LCR
Lenalidomide
Letrozole
Leucovorin
Leukeran
Leukine ™
Leuprolide
Leurocristine
Leustatin ™
Liposomal Ara-C
Liquid Pred ®
Lomustine
L-PAM
L-Sarcolysin
Lupron ®
Lupron Depot ®
M
Matulane ®
Maxidex
Mechlorethamine
Mechlorethamine Hydrochloride
Medralone ®
Medrol ®
Megace ®
Megestrol
Megestrol Acetate
Melphalan
Mercaptopurine
Mesna
Mesnex ™
Methotrexate
Methotrexate Sodium
Methylprednisolone
Meticorten ®
Mitomycin
Mitomycin-C
Mitoxantrone
M-Prednisol ®
MTC
MTX
Mustargen ®
Mustine
Mutamycin ®
Myleran ®
Mylocel ™
Mylotarg ®
N
Navelbine ®
Nelarabine
Neosar ®
Neulasta ™
Neumega ®
Neupogen ®
Nexavar ®
Nilandron ®
Nilotinib
Nilutamide
Nipent ®
Nitrogen Mustard
Novaldex ®
Novantrone ®
Nplate
O
Octreotide
Octreotide acetate
Ofatumumab
Oncospar ®
Oncovin ®
Ontak ®
Onxal ™
Oprelvekin
Orapred ®
Orasone ®
Oxaliplatin
P
Paclitaxel
Paclitaxel Protein-bound
Pamidronate
Panitumumab
Panretin ®
Paraplatin ®
Pazopanib
Pediapred ®
PEG Interferon
Pegaspargase
Pegfilgrastim
PEG-INTRON ™
PEG-L-asparaginase
PEMETREXED
Pentostatin
Phenylalanine Mustard
Platinol ®
Platinol-AQ ®
Prednisolone
Prednisone
Prelone ®
Procarbazine
PROCRIT ®
Proleukin ®
Prolifeprospan 20 with Carmustine Implant
Purinethol ®
R
Raloxifene
Revlimid ®
Rheumatrex ®
Rituxan ®
Rituximab
Roferon-A ® (Interferon Alfa-2a)
Romiplostum
Rubex ®
Rubidomycin hydrochloride
S
Sandostatin ®
Sandostatin LAR ®
Sargramostim
Solu-Cortef ®
Solu-Medrol ®
Sorafenib
SPRYCEL ™
STI-571
Streptozocin
SU11248
Sunitinib
Sutent ®
T
Tamoxifen
Tarceva ®
Targretin ®
Tasigna ®
Taxol ®
Taxotere ®
Temodar ®
Temozolomide
Temsirolimus
Teniposide
TESPA
Thalidomide
Thalomid ®
TheraCys ®
Thioguanine
Thioguanine Tabloid ®
Thiophosphoamide
Thioplex ®
Thiotepa
TICE ®
Toposar ®
Topotecan
Toremifene
Torisel ®
Tositumomab
Trastuzumab
Treanda ®
Tretinoin
Trexall ™
Trisenox ®
TSPA
TYKERB ®
V
VCR
Vectibix ™
Velban ®
Velcade ®
VePesid ®
Vesanoid ®
Viadur ™
Vidaza ®
Vinblastine
Vinblastine Sulfate
Vincasar Pfs ®
Vincristine
Vinorelbine
Vinorelbine tartrate
VLB
VM-26
Vorinostat
Votrient
VP-16
Vumon ®
X
Xeloda ®
Z
Zanosar ®
Zevalin ™
Zinecard ®
Zoladex ®
Zoledronic acid
Zolinza
Zometa ®

As used herein a “therapeutic effect” should be taken to mean, at a minimum, a reduction of or remission from the symptoms or effects of the underlying disease or condition. In a ‘best case’ scenario, the therapeutic effect may be manifested by a complete termination, either temporary or permanent, of such symptoms or effects. Thus as also used herein a “treatment” should be taken to mean a step that produces such a therapeutic effect. As further used herein, a “therapeutic amount” should be taken to mean an amount or dosage, e.g., of the chemotherapeutic agent, sufficient to obtain a desirable therapeutic effect. It would be well within the capability of one having at least an ordinary level of skill in the relevant field of art to determine the parameters of both a therapeutic amount and a therapeutic effect for a particular medicament falling within the scope of the methods and formulations described and claimed herein and/or to identify a disease or condition of the type that may be treated with such methods/formulations. As used herein, facilitating entry of the chemotherapeutic agent into cells of an individual afflicted with a disease or condition is meant to refer to rendering it easier for the agent to enter the cell through the cell wall and/or by permitting a greater amount of the chemotherapeutic agent to enter the cell than would otherwise occur without the presence of the at least one phospholipid or the composition (including but not limited to lecithin) containing at least one phospholipid.

As used herein, cancer refers to the uncontrolled growth of abnormal cells in an individual's body. Cancer cells are often referred to as “malignant cells”. The presently disclosed methods and formulations are not limited to treatment of cancer, but rather cancers are predominantly discussed herein as they provide a ‘typical’ condition involving treatment with chemotherapeutic agents and due to the fact that the inventor of the claimed methods and formulations has himself been afflicted with a particular hematological malignancy, i.e., multiple myeloma. As used herein a “hematological malignancy” is meant to refer to the types of cancer that effect blood, bone marrow and lymph nodes. Multiple myeloma as discussed herein is a cancer of plasma cells, i.e., a type of white blood cell normally responsible for the production of antibodies. In the course of the disease, collections of abnormal cells accumulate in bones where they cause bone lesions (abnormal areas of tissue) and in the bone marrow where they interfere with the production of normal blood cells.

Lecithin as noted above, is a useful source, although not the only source, of phospholipids useful in carrying out the method described herein. Although lecithin is considered to be of low toxicity, anyone who is allergic to products derived from soy should exercise caution before using it. The method as described herein is not limited to the administration of lecithin, however, since as would be well known by those having an ordinary level of skill in this field, there are numerous sources of phospholipids that may be administered in place of, or together with, lecithin in the present method. It is not the source of the phospholipid that is important here. Rather, any and all phospholipids that operate pursuant to the mechanism as described above, i.e., whereby the material acts as a dispersing agent/emulsifier, and serves to facilitate access to cells, i.e., particularly abnormal cells (e.g., cancer cells) by easing passage of one or more chemotherapeutic agents through the cells walls, is considered to be within the scope of the formulations and methods described herein.

As mentioned elsewhere herein, lecithin is found naturally in the yolk of eggs. However, lecithin is also added to some other products such as, for example, chocolate bars where the material serves as an emulsifier. Analogously in the case of the presently claimed formulations and methods, lecithin or a component thereof—such as a phospholipid—may be used either individually with chemotherapy or in intimate combination with such chemotherapy. In the latter case, the combined composition may comprise part or all of any convenient form including, but not limited to, tablets, capsules, vegetable capsules and/or a liquid solution for drinking or for introduction into an infusion.

The efficacy of the presently described composition and method is demonstrated by the following example. A patient who had been diagnosed with asymptomatic myeloma progressed to a diagnosis of full blown multiple myeloma, and began to receive chemotherapy. Upon receiving the diagnosis of multiple myeloma the patient ceased taking various vitamins and supplements with the exception of (1) lecithin, a tablespoon of which granules were sprinkled each morning on his breakfast cereal on a daily basis and (2) aspirin, which was prescribed in order to mitigate the risk of developing blood clots as a consequence of taking thalidomide as a part of the prescribed chemotherapy. Various chemotherapy drugs were administered to the patient in concert with the lecithin, including cyclophosphamide, dexamethasone, bortezomib and pamidronate. These agents were administered in various dosages and at various time intervals. While not wishing to be bound by theory, it is believed that the method and compositions disclosed herein should prove useful with a wide variety of chemotherapeutic agents since the method is not dependent upon the agent itself but rather upon he mechanism for facilitating entry of the agents into the cells of an individual who is subject to the method.

As shown in Table 2 below, at the beginning of the chemotherapeutic treatment described above, on May 27, 2011 the patient's IG G immunoglobulins were abnormally high (8479) and his other immunoglobulins, i.e., IG M (18) and IGA (33) were abnormally low. The patient's M-spike % was 55.70 and his free lambda serum was an extremely high 12364.50. However, by Jul. 14, 2011, the patient's IG G had plunged to 922, within the normal range, and the patient's free lambda serum was only 9.19, also within the normal range. Furthermore, by Aug. 25, 2011, the patient's blood had no detectable M-spike, and by Sep. 29, 2011, the patient's IG M and IGA immunoglobulins were also in the normal range. A summary of the patient's cancer panel blood test results for the period May 27, 2011 through Sep. 29, 2011, containing information provided by the patient's physician, is set forth in Table 2. The patient was then in complete remission.

TABLE 2

| | 1 | 2 | 3 | 4 | 5 | 6 | 7 |
|---|---|---|---|---|---|---|---|
| | May 27, 2011 | Jun. 16, 2011 | Jul. 14, 2011 | Jul. 22, 2011 | Aug. 18, 2011 | Aug. 25, 2011 | Sep. 29, 2011 |
| IG G QUANT | 8479 | 3443 | 922 | 784 | | 673 | 779 |
| IG M QUANT | 18 | 71 | 34 | 33 | | 38 | 145 |
| IG A QUANT | 33 | 74 | 41 | 56 | | 75 | 157 |
| M-SPIKE % | 55.70 | 40.60 | 11.60 | 7.20 | | None | cancelled |
| M-SPIKE G/DL | 7.29 | 2.96 | 0.56 | 0.35 | | None | cancelled |
| FREE KAPPA SERUM | 11.93 | 29.16 | 7.52 | 12.73 | 15.52 | 17.49 | 31.97 |
| FREE LAMBDA | 12364.50 | 260.91 | 9.19 | 12.07 | 15.18 | 18.56 | 20.95 |
| SERUM | | | | | | | |
| FREE KAPPA/ | 0.001 | 0.112 | 0.818 | 1.055 | 1.022 | 0.942 | 1.526 |
| LAMBDA | | | | | | | |

Although a small percentage of multiple myeloma patients may achieve complete remission without receiving a bone marrow transplant, the patient described herein not only did achieve such complete remission, but also did so in a remarkably short period of time. In view of the fact that lecithin was the only dietary supplement being taken by the patient during the period he received chemotherapy, it is reasonable to infer that his daily ingestion of lecithin, while not in itself a cure, served as an aid toward such a cure or at least a temporary complete remission by enhancing the efficiency of the chemotherapy in destroying abnormal plasma cells in the patient's bone marrow or wherever located. Assuming further that this conclusion is correct, it is also reasonable to expect that lecithin ingestion will improve results of the other chemotherapies for multiple myeloma, and for diseases other than multiple myeloma.

The amount of lecithin required may be any effective amount, and may vary from less than a teaspoon daily up to two tablespoons daily. However, excessively large doses should be avoided.

Furthermore, although the aforesaid outstanding results were achieved through the use of lecithin, as indicated above an alternative phospholipid-containing compound, or phospholipid by itself could be used instead of lecithin provided that such material used is also compatible with a patient's cell membranes.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A method for improving efficacy of a chemotherapeutic treatment of a disease or condition, wherein the method comprises:
(a) administering a therapeutic amount of at least one chemotherapeutic agent to an individual afflicted with a disease or condition that is treatable by said at least one agent; and
(b) also administering to the individual at least one phospholipid or a material containing at least one said phospholipid, wherein said at least one phospholipid is administered in an amount sufficient to facilitate entry of said at least one chemotherapeutic agent into cells of said individual negatively affected by the disease or condition,
wherein steps (a) and (b) are performed in any order, but in sufficient time proximity such that the effect of the at least one phospholipid in facilitating entry of said at least one chemotherapeutic agent into cells of said afflicted individual at least partially overlaps with the therapeutic effect of said at least one chemotherapeutic agent.

2. The method of claim 1, wherein the material containing said at least one phospholipid is lecithin.

3. The method of claim 1, wherein the disease or condition is a cancer susceptible to treatment by said at least one chemotherapeutic agent.

4. The method of claim 3, wherein the cancer is a hematological malignancy.

5. The method of claim 4, wherein the hematological malignancy is multiple myeloma.

6. The method of claim 1, which further comprises administering a sufficient dosage of a blood-thinning agent to said individual to mitigate risk of developing blood clots attributable to administration of the at least one chemotherapeutic agent.

7. The method of claim 6, wherein the blood-thinning agent is aspirin or pycnogenol.

8. The method of claim 1, wherein the at least one phospholipid or the material containing said at least one phospholipid is combined with the at least one chemotherapeutic agent in a dosage form selected from the group consisting of tablets, capsules, vegetable capsules, an injectable, a trans-dermal patch and liquid solutions formulated for drinking or for infusion.

9. The method of claim 6, wherein the at least one phospholipid or the material containing said at least one phospholipid is combined with the at least one chemotherapeutic agent and the blood-thinning agent in a dosage form selected from the group consisting of tablets, capsules, vegetable capsules, an injectable, a trans-dermal patch and liquid solutions formulated for drinking or for infusion.

10. A method for improving efficacy of a chemotherapeutic treatment of a disease or condition, wherein the method comprises:
(a) administering a therapeutic amount of at least one chemotherapeutic agent to an individual afflicted with a disease or a condition that is treatable by said at least one agent;
(b) also administering to the individual at least one phospholipid or a material containing at least one said phospholipid, wherein the at least one phospholipid is administered in an amount sufficient to facilitate entry of said at least one chemotherapeutic agent into cells of said individual negatively affected by the disease or condition; and
(c) further administering a sufficient dosage of a blood-thinning agent to the individual to mitigate risk of developing blood clots attributable to administration of the at least one chemotherapeutic agent,
wherein steps (a), (b) and (c) are performed in any order but in sufficient time proximity such that the effect of the at least one phospholipid in facilitating entry of said at least one chemotherapeutic agent into cells of said afflicted individual at least partially overlaps with the therapeutic effect of said at least one chemotherapeutic agent.

11. The method of claim 10, wherein the material containing at least one said phospholipid is lecithin.

12. The method of claim 11, wherein the disease or condition is a cancer susceptible to treatment by said at least one chemotherapeutic agent.

13. The method of claim 12, wherein the cancer is a hematological malignancy.

14. The method of claim 13, wherein the hematological malignancy is multiple myeloma.

15. The method of claim 10, wherein the blood-thinning agent is aspirin or pycnogenol.

16. The method of claim 10, wherein the at least one phospholipid or the material containing said at least one phospholipid is combined with the at least one chemotherapeutic agent and the blood-thinning agent in a dosage form selected from the group consisting of tablets, capsules, vegetable capsules, an injectable, a trans-dermal patch and liquid solutions formulated for drinking or for infusion.

17. A formulation that improves efficacy of chemotherapeutic treatment of an individual afflicted with a condition or a disease treatable with one or more chemotherapeutic agents, said formulation comprising:
(a) at least one chemotherapeutic agent capable of treating a disease or condition with which said individual is afflicted; and
(b) at least one phospholipid or a material containing at least one said phospholipid, wherein the at least one phospholipid acts to facilitate entry of the at least one chemotherapeutic agent into cells of said individual,
wherein the at least one chemotherapeutic agent and the at least one phospholipid or the material containing said at least one phospholipid are administered in any order, but in sufficient time proximity such that the effect of the at least one phospholipid of facilitating entry of said at least one chemotherapeutic agent into cells of said afflicted individual at least partially overlaps with the therapeutic effect of said at least one chemotherapeutic agent.

18. The formulation of claim 17, wherein the material containing said at least one phospholipid is lecithin.

19. The formulation of claim 17, further comprising a blood-thinning agent.

20. The formulation of claim 19, wherein the blood-thinning agent is aspirin or pycnogenol.

21. The formulation of claim 17, wherein said formulation is provided in a dosage form selected from the group consisting of tablets, capsules, vegetable capsules, an injectable, a trans-dermal patch and liquid solutions formulated for drinking or for infusion.

22. A formulation that improves efficacy of chemotherapeutic treatment of an individual afflicted with a condition or a disease treatable with one or more chemotherapeutic agents, said formulation comprising:
(a) at least one chemotherapeutic agent capable of treating a disease or condition with which said individual is afflicted;
(b) at least one phospholipid or a material containing said at least one phospholipid, wherein the at least one phospholipid acts to facilitate entry of the at least one chemotherapeutic agent into cells of said individual; and
(c) a sufficient dosage of a blood-thinning agent to mitigate risk to the individual of developing blood clots attributable to administration of the at least one chemotherapeutic agent,
wherein the at least one chemotherapeutic agent and the at least one phospholipid or the material containing said at least one phospholipid are administered in any order, but in sufficient time proximity such that the effect of the at least one phospholipid of facilitating entry of said at least one chemotherapeutic agent into cells of said afflicted individual at least partially overlaps with the therapeutic effect of said at least one chemotherapeutic agent.

23. The formulation of claim 22, wherein the material containing said at least one phospholipid is lecithin.
